(19) United States
(12) Patent Application Publication
(10) Pub. No.: US 2025/0125011 A1
(43) Pub. Date: Apr. 17, 2025

(54) SYSTEMS AND METHODS FOR INTELLIGENT CONSTRUCTION OF ANTIBODY LIBRARIES

(71) Applicant: Adimab, LLC, Lebanon, NH (US)

(72) Inventors: Tushar Jain, Lebanon, NH (US); Maximiliano Vásquez, Lebanon, NH (US); Kyle Andrew Barlow, Lebanon, NH (US)

(21) Appl. No.: 18/706,089

(22) PCT Filed: Oct. 26, 2022
(86) PCT No.: PCT/US2022/047888

§ 371 Date: Apr. 30, 2024

Related U.S. Application Data
(60) Provisional application No. 63/274,394, filed on Nov. 1, 2021.

(51) Int. Cl. G16B 35/00; G06N 3/09; G16B 15/20; G16B 15/30; G16B 40/20

(52) U.S. Cl. CPC G16B 35/00; G06N 3/09; G16B 15/20; G16B 15/30; G16B 40/20

(57) ABSTRACT

Presented herein are systems and methods for constructing antibody libraries using machine learning to inform sequence selection for inclusion in the library. The techniques include (i) the training and use of machine learning models and statistical models to predict biophysical and biochemical properties from sequences, and (ii) the training and use of machine learning models for predicting developability from sequences, and for generating novel sequences. In certain embodiments, the systems and methods generate libraries of antibodies (and/or antibody-encoding polynucleotides) by specifically designing the libraries with directed sequence and/or length diversity. The resulting libraries are useful, for example, in the development of therapeutic agents.

### CROSS REFERENCE TO RELATED APPLICATIONS

This application claims priority to and the benefit of U.S. Provisional Patent Application No. 63/274,394, filed Nov. 1, 2021, the entirety of which is incorporated herein by reference.

### BACKGROUND

Antibodies have profound relevance as research tools and in diagnostic and therapeutic applications. However, the identification of useful antibodies is difficult and once identified, antibodies often require considerable redesign before they are suitable for human therapeutic applications.

Accordingly, a need exists for smaller (i.e. able to be synthesized and physically realizable) antibody libraries with directed diversity that systematically represent candidate antibodies that are non-immunogenic (e.g., more human) and have desired properties, such as, for example, the ability to recognize a broad variety of antigens. Obtaining such libraries requires balancing the competing objectives of restricting the sequence diversity represented in the library (e.g., to enable synthesis and physical realization, potentially with oversampling, while limiting the introduction of non-human sequences) while maintaining a level of diversity sufficient to recognize a broad variety of antigens.

Thus, a need exists for methods of constructing antibody libraries populated with antibodies which (a) can be readily synthesized, (b) can be physically realized and, in certain cases, oversampled, (c) contain sufficient diversity to recognize all antigens recognized by the preimmune human repertoire (i.e., before negative selection), (d) are non-immunogenic in humans (i.e. comprise sequences of human origin), and/or (e) contain CDR length and sequence diversity, and framework diversity, representative of naturally-occurring human antibodies.

### SUMMARY

Presented herein are systems and methods for constructing antibody libraries using machine learning to inform sequence selection for inclusion in the library. The techniques include (i) the training and use of machine learning models and statistical models to predict biophysical and biochemical properties from sequences, and (ii) the training and use of machine learning models for predicting developability from sequences, and for generating novel sequences. In certain embodiments, the systems and methods generate libraries of antibodies (and/or antibody-encoding polynucleotides) by specifically designing the libraries with directed sequence and/or length diversity. The resulting libraries are useful, for example, in the development of therapeutic agents.

In one aspect, the invention is directed to a system for constructing (e.g., designing) an antibody library, the system comprising: a processor of a computing device; and a memory having instructions stored thereon, wherein the instructions, when executed by the processor, cause the processor to perform one or more of (i), (ii), (iii), (iv), (v), (vi), and (vii) as follows: (i) develop (e.g., train) a first machine learning model using input sequences and characterization data [e.g., (a) train a logistic regression model to derive amino-acid coefficients to predict poly-specificity and hydrophobicity for individual complementarity-determining regions (CDRs) and/or framework regions (FRs); and/or (b) train a tree-based model (e.g., randomforest or XGBoost) to predict one or more biophysical properties and/or one or more chemical stability properties from a sequence; and/or (c) train a deep-learning model comprising neural networks to predict one or more biophysical properties and/or one or more chemical stability properties from a sequence (e.g., wherein the model comprises an input layer, multiple intermediate feature-extraction layers, and a final output layer); and/or (d) create a statistical model to evaluate bias to select sequences with low bias; and/or (e) develop hierarchical statistics to predict risk of chemical modification as a function of sequence motifs at a specific position and region (e.g., H1, H2, H3, L1, L2, L3, HFR, LFR)]; (ii) use the first machine learning model in (i) to predict desirable segments (e.g., segments with favorable predicted expression enrichment) to enable selection of segments from a pool of de novo and/or pre-generated segments; (iii) process a set of input sequences prior to selection and/or use in training the first machine learning model in (i), wherein processing the set of input sequences comprises one or more of: (a) eliminating chemical liability sites by modifying the sequence, (b) for CDR H3, splitting the sequence into segments to mimic VDJ recombination, (c) for CDR L3, splitting the sequence into segments to mimic VJ recombination, and (d) annotating V-regions and CDRs (H1, H2, L3) with number of mutations from germline; (iv) train a machine learning model for biophysical and/or biochemical property prediction (e.g., using data on a set of input sequences sorted for favorable biophysical properties, e.g., low poly-specificity, low hydrophobicity, and/or high expression); (v) use the machine learning model for biophysical and/or biochemical property prediction in (iv) to predict one or more biophysical and/or biochemical properties from a sequence (e.g., poly-specificity, hydrophobicity, melting temperature, SEC monomer percentage, retention time, chemical stability data, and/or a measure of sequence enrichment or depletion; (vi) develop (e.g., train) an auto-regressive deep-learning neural network model to learn a joint sequence probability distribution over sequences of interest for specific germlines for different species; and (vii) use the neural network model in (vi) to capture sequence compositions and/or correlations from an input set of sequences and produce novel sequences or segments for consideration in a synthetic library.

In another aspect, the invention is directed to a system for constructing an antibody library, the system comprising: a processor of a computing device; and a memory having instructions stored thereon, wherein the instructions, when executed by the processor, cause the processor to process a set of input sequences to generate a collection of final antibody library sequences using one or more machine learning models.

In certain embodiments, the instructions cause the processor to process (i) each input sequence from the set of input sequences as well as, (ii) for each of the input sequences, per-residue predictions of one or more structurally-relevant properties of the sequence as predicted by a first model (e.g., a graph convolutional network (GCN)), said instructions causing the processor to process (i) and (ii) as input in a second model to predict, as output of the second model, (iii) one or more biophysical properties [e.g., hydrophobic interaction chromatography retention time (HIC RT) and/or polyspecificity reagent (PSR) score and/or PSR binding category] and/or (iv) one or more chemical stability properties [e.g., Asn deamidation, Asp isomerization, and/or Met oxidation] of each of the input sequences, wherein inclusion or exclusion of each sequence in the final antibody library is based at least in part on the output of the second model.

In certain embodiments, the per-residue predictions predicted by the first model comprise one or more members selected from the group consisting of (i) a measure of solvent accessibility (SASA), (ii) a measure of charge patches, (iii) a measure of hydrophobic patches, and (iv) Cα/Cβ coordinate predictions.

In certain embodiments, the second model comprises a deep convolution and/or recurrent network (e.g., for prediction of biophysical properties).

In certain embodiments, the second model comprises a tree-based classification model (e.g., for prediction of chemical stability).

In another aspect, the invention is directed to a method for constructing (e.g., designing) an antibody library, the method comprising using a processor of a computing device to perform one or more of (i), (ii), (iii), (iv), (v), (vi), and (vii) as follows: (i) developing (e.g., training) a first machine learning model using input sequences and characterization data [e.g., (a) training a logistic regression model to derive amino-acid coefficients to predict poly-specificity and hydrophobicity for individual complementarity-determining regions (CDRs) and/or framework regions (FRs); and/or (b) training a tree-based model (e.g., randomforest or XGBoost) to predict one or more biophysical properties and/or one or more chemical stability properties from a sequence; and/or (c) training a deep-learning model comprising neural networks to predict one or more biophysical properties and/or one or more chemical stability properties from a sequence (e.g., wherein the model comprises an input layer, multiple intermediate feature-extraction layers, and a final output layer); and/or (d) creating a statistical model to evaluate bias to select sequences with low bias; and/or (e) developing hierarchical statistics to predict risk of chemical modification as a function of sequence motifs at a specific position and region (e.g., H1, H2, H3, L1, L2, L3, HFR, LFR)]; (ii) using the first machine learning model in (i) to predict desirable segments (e.g., segments with favorable predicted expression enrichment) to enable selection of segments from a pool of de novo and/or pre-generated segments; (iii) processing a set of input sequences prior to selection and/or use in training the first machine learning model in (i), wherein processing the set of input sequences comprises one or more of: (a) eliminating chemical liability sites by modifying the sequence, (b) for CDR H3, splitting the sequence into segments to mimic VDJ recombination, (c) for CDR L3, splitting the sequence into segments to mimic VJ recombination, and (d) annotating V-regions and CDRs (H1, H2, L3) with number of mutations from germline; (iv) training a machine learning model for biophysical and/or biochemical property prediction (e.g., using data on a set of input sequences sorted for favorable biophysical properties, e.g., low poly-specificity, low hydrophobicity, and/or high expression); (v) using the machine learning model for biophysical and/or biochemical property prediction in (iv) to predict one or more biophysical and/or biochemical properties from a sequence (e.g., poly-specificity, hydrophobicity, melting temperature, SEC monomer percentage, retention time, chemical stability data, and/or a measure of sequence enrichment or depletion); (vi) developing (e.g., training) an auto-regressive deep-learning neural network model to learn a joint sequence probability distribution over sequences of interest for specific germlines for different species; and (vii) using the neural network model in (vi) to capture sequence compositions and/or correlations from an input set of sequences and produce novel sequences or segments for consideration in a synthetic library.

In another aspect, the invention is directed to a method for constructing (e.g., designing) an antibody library, the method comprising: processing, by a processor of a computing device, a set of input sequences to generate a collection of final antibody library sequences using one or more machine learning models.

In certain embodiments, the method comprises processing, as input in a second model, (i) each input sequence from the set of input sequences as well as (ii) for each of the input sequences, per-residue predictions of one or more structurally-relevant properties of the sequence as predicted by a first model (e.g., a graph convolutional network (GCN)), to produce, as output of the second model, (iii) one or more biophysical properties [e.g., hydrophobic interaction chromatography retention time (HIC RT) and/or polyspecificity reagent (PSR) score and/or PSR binding category] and/or (iv) one or more chemical stability properties [e.g., Asn deamidation, Asp isomerization, and/or Met oxidation] of each of the input sequences, wherein inclusion or exclusion of each sequence in the final antibody library is based at least in part on the output of the second model.

In certain embodiments, the per-residue predictions predicted by the first model comprise one or more members selected from the group consisting of (i) a measure of solvent accessibility (SASA), (ii) a measure of charge patches, (iii) a measure of hydrophobic patches, and (iv) Cα/Cβ coordinate predictions.

In certain embodiments, the second model comprises a deep convolution and/or recurrent network (e.g., for prediction of biophysical properties).

In certain embodiments, the second model comprises a tree-based classification model (e.g., for prediction of chemical stability).

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1 is a block flow diagram of an illustrative method for informed construction of antibody sequence libraries, according to an illustrative embodiment.

FIG. 2 is a schematic diagram of a deep-learning module for predicting developability from sequences, according to an illustrative embodiment.

FIG. 3 is a chart showing an example for matching a particular CDR H3 sequence in a VHH H3 library design, according to an illustrative embodiment.

FIG. 4A is a diagram illustrating an example sequence generation procedure used in CDRs H1 and H2 library design, according to an illustrative embodiment.

FIG. 4B is a diagram illustrating an example sequence generation procedure used in Vλ L3 library design, according to an illustrative embodiment.

FIG. 5 is a chart showing an example distribution used for calculating KL(L91A, Vκ1-39) in a method for Vκ L3 sequence design, according to an illustrative embodiment.

FIG. 6 is a schematic diagram of a network environment for use in providing systems, methods, and architectures described herein.

FIG. 7 is a schematic diagram depicting a computing device and a mobile computing device that can be used to implement the techniques described herein.

FIGS. 8A and 8B are schematic diagrams that show steps in an illustrative machine learning method for predicting structural properties from sequence data.

FIG. 9 is a block diagram of a method of using predictions of structurally relevant metrics for biophysical properties in a model to predict key developability properties for therapeutics, according to an illustrative embodiment.

FIGS. 10A and 10B are schematic diagrams illustrating the use of graphical convolution models for residue level prediction of structural descriptors, according to an illustrative embodiment.

FIG. 11 is a graph showing overall SAP score calculated by summing individual residue predictions according to an illustrative embodiment, where the predictions are comparable to those obtained from AlphaFold2 models for the same input sequences.

FIG. 12 are graphs showing overall SCM score calculated by summing individual residue predictions, according to an illustrative embodiment.

FIG. 13 is a schematic of Mollweide projections of predicted net charge patches, according to an illustrative embodiment.

FIG. 14 is a schematic illustrating the use of convolution models and recurrent models for prediction of hydrophobicity and polyspecificity, according to an illustrative embodiment.

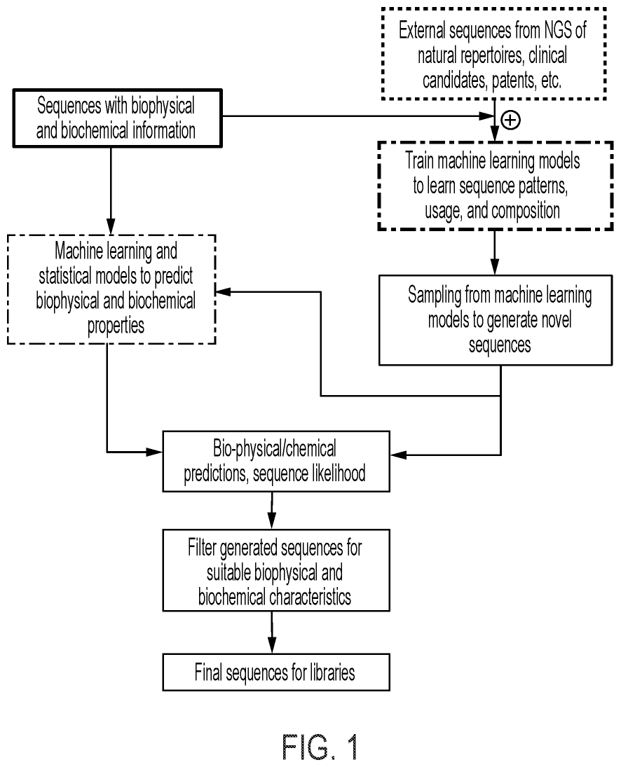

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents referenced herein are incorporated herein by reference. Where there is any discrepancy in the meaning of a particular term, the meaning provided in the Detailed Description section is controlling.

Headers are provided for the convenience of the reader—the presence and/or placement of a header is not intended to limit the scope of the subject matter described herein.

### Section I. Training Sequence Sets for Learning Sequence Patterns, Correlations, and Usage in Natural or Synthetic Repertoires

Sequences used in the systems and methods presented herein can come from internal discovery (naïve, LCBS [light chain batch shuffle], premade AFFMAT [affinity maturation], oligo-based lead-specific AFFMAT), patents and clinical sequences, or literature NGS [next generation sequencing] datasets. Once the starting set of sequences is obtained, they can be post-processed depending on the nature of the library and the CDR [complementarity-determining regions]. A list of illustrative post-processing steps includes:
1) Eliminating chemical liability sites by modifying the sequence in the following manner:
   a. Replace exposed Met with Leu;
   b. Replace N[G,S,T] with Q[G,S,T], thus removing potential Asn deamidation motifs;
   c. Replace D[G,S,T] with E[G,S,T], which removes Asp isomerization motifs;
   d. Replace Asn in N-gly sites with Asp and thus remove N-linked glycosylation motifs, which are deemed as potential liabilities due to host cell-dependence, among other factors;
   e. Replace fragmentation motif DP with EP; and
   f. Replace N, D, or M amino acids predicted (by trained machine learning models) to be at high risk of modification, or mutate their surrounding sequence context, to reduce modification risk (as also predicted by machine learning models).
2) For CDR H3, split the sequence into segments to mimic VDJ recombination:
   a. Segments could be based on matching to pre-generated libraries of segments from known V, D, and J genes;
   b. Inferred from parsing and analyzing output of programs such as IgBlast, Immcantation [Vander Heiden J A, Yaari G, Bioinformatics, 30, 1930, 2014 PMID: 24618469; Gupta N T, Vander Heiden J A, Bioinformatics, 31, 3356, 2015, PMID: 26069265], and the like; and
   c. Inferred de novo from in house software;
3) For CDR L3, split the sequence into segments to mimic VJ recombination:
   a. Segments are based on matching to known V- and J-genes from literature or IMGT database;
4) Additionally, annotate V-regions and CDRs (H1, H2, L3) with:
   a. Number of mutations from germline; and
   b. Number of mutations from germline for the preferentially antigen contacting or exposed residues based on analysis of published crystal structures.

Examples of sequences used to train models for different library designs include the following:
1) Variable region sequence data for human germlines:
   a) From OAS (Observed Antibody Space) sequence database [Kovaltsuk A, The Journal of Immunology, 201, 2502, 2018, PMID: 30217829];
   b) Internal data from human germlines from primary discovery and affinity maturation with paired heavy and light chain sequences; and
   c) Clinical antibody data from literature, patents, etc. with paired heavy and light chain sequences;
2) Human V, D, and J-gene information from IMGT;
3) Variable region sequence data for camelidae from NGS:
   a) Llama sequences from McCoy L E, PLOS Pathogens, 10, e1004552, 2014, PMID: 25522326;
   b) Bactrian camel sequences from Li X, PLOS ONE, 11, e0161801, 2016, PMID: 27588755; and
   c) Clinical antibody data from literature, patents, and the like.
4) Camelid V, D, and J-gene information:
   a) V-genes from IMGT for alpaca, llama, and bactrian camel;
   b) J-genes from IMGT for alpaca, llama, and from Liang Z, Frontiers of Agricultural Science and Engineering, 2, 249, 2015 for bactrian camel; and
   c) D-genes from IMGT for alpaca, and internally inferred from NGS data using IgScout [Safonova Y, Frontiers in Immunology, 10, 1, 2019, PMID: 31134072] for llama and bactrian camel, and Liang Z, Frontiers of Agricultural Science and Engineering, 2, 249, 2015 for bactrian camel.

### Section II. Machine Learning for Predicting Structural Properties Directly from Sequence

Because obtaining 3D structures, either from experimental techniques such as X-ray crystallography, cryo-EM, or from homology modeling software like AlphaFold, IgFold, or Schrodinger Discovery Studio can be time consuming, presented herein are machine learning models to predict structural properties important for downstream developability predictions from sequence input.
1) 3D structure data for developing the machine model(s) may be obtained from:
   a) Publicly deposited or internally obtained Protein Data Bank (PDB) structures; and/or
   b) Homology models from publicly available sources or generated via internal software pipelines and algorithms.
2) Internally developed proprietary algorithms or internal implementations of published methods (e.g., S A P Chennamsetty et al., J. Phys. Chem., 2014, S C M Agrawal et al., mAbs, 2016), are used with the above 3D structure data to generate descriptors for each residue in the input structure. Additionally, values of these descriptors can be aggregated based on residue-type, antibody region, or combinations of the above to generate higher level descriptors.

The sequences of the 3D structures in item 1) serve as input data to machine learning models that aim to predict a set of descriptors in item 2) above.

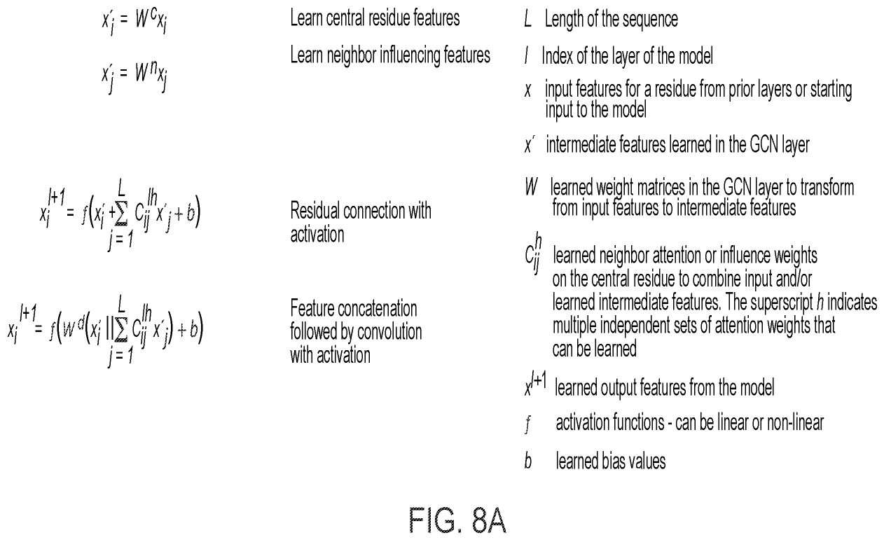

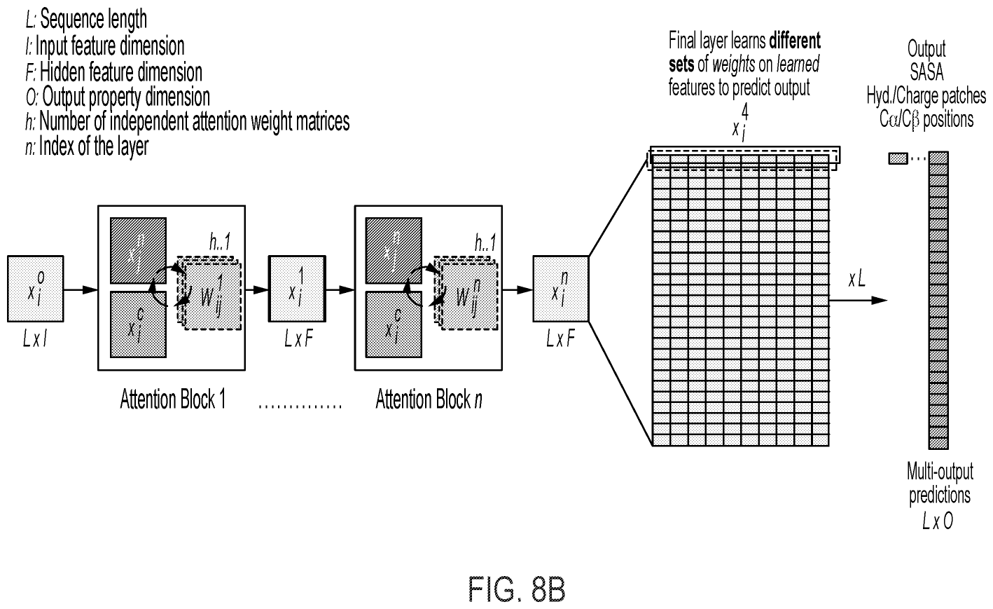

FIGS. 8A and 8B show steps in an illustrative machine learning method for predicting structural properties from sequence data.

Since protein structures can be represented as graphs, a graphical convolution network (GCN) architecture may be used to predict structural properties from sequence. The GCN includes the following operations:
1) Networks weights, W, are learned individually for each amino-acid type, position or a combination thereof—so called node weights; and
2) Weights are also learned to represent the effect of neighboring residues on a central residue Cij—so-called edge weights;
3) The node and edge weights can be combined via mathematical operations denoted by f, including bias terms b, to generate descriptors or additional features for each residue in the sequence, denoted by x;
4) To increase the learning capacity of the network, and enable it to learn properties over multiple length scales, an independent set of the above parameters can be learned at each step;
5) To enable complex relationships to be learned by the network, multiple such layers can be stacked to construct a deep learning model. Each layer is denoted as an “Attention Block” in the schematic of FIG. 8B; and
6) A dense layer with non-linear activations is implemented where position specific weights are learned in order to finally predict the structural descriptors for each residue.

Examples of structural descriptors that can be learned by the model and subsequently predicted with only sequence as the input are as follows:
1) Solvent accessibility for each residue;
2) Extent of hydrophobicity around each residue, calculated using a set of published or determined hydrophobicity/hydrophilicity propensities, and over multiple length scales;
3) Extent of positive, negative, and overall charge around each residue, calculated using charges assigned from difference forcefields such as CHARMM, AMBER, calculated at different pHs, and over multiple length scales; and
4) Calculation of structural coordinates for the backbone and side-chain obtained after aligning the input training data to a common reference frame.

The prediction of these descriptors from sequence can then serve as input to downstream tasks and other machine learning models to predict experimentally observed developability properties for antibodies.

Sequences for the input structures were aligned using a consistent numbering scheme and converted into numbers using a one-hot encoding scheme, addition of biophysical and biochemical features using amino-acid property scales, position-specific scoring matrices, and pre-trained sequence embeddings.

A 25-fold Monte-Carlo cross-validation was used to train the models with training and validation splits of 80% and 20%, respectively. Model training was carried out for a maximum of 200 epochs with early termination if improvement was not seen on the test set for more than 10 epochs.

Since the output descriptors to be predicted have different scales of values, a preprocessing step can be performed such that the distribution for each descriptor is centered by subtracting the mean on a per-residue basis. Furthermore, different strategies for scaling the magnitudes were employed, such as dividing by the variance or interquartile ranges of the original distribution.

Illustrative pseudo-code for deep-learning models to predict structural descriptors from sequence is as follows:

| | |
|---|---|
| 1. | xᵢ = Concatenate[one - hot encoding, amino - acid property |
| | scales, pre - trained embeddings, position - |
| | specific scoring matrix, multiple sequence alignment] |
| 2. | Eᵢ = EmbeddingLayer(xᵢ) |
| 3. | xᵢ⁰ = Eᵢ |
| | For k in 1:NumAttentionBlocks: |
| | xᵢ^{k} = GraphConvolutionBlockₖ(x^{k-1}ᵢ) |
| 4. | Cᵢ = xᵢ^{k} |
| | For c in 1: NumConvolutionLayers: |
| | Cᵢ = ConvolutionLayer_{c}(Cᵢ) |
| 5. | Dᵢ = Cᵢ |
| | For d in 1:NumDenseLayers: |
| | Dᵢ = Dense_{d}ⁿ(Dᵢ) |
| 6. | Oᵢⁿ = Denseⁿ(Dᵢ) |

i ∈ [1 ... L], where L is the sequence length
n ∈ [1 ... N], where n indexes the N output structural descriptors to be
predicted

### Section III. Input Training Data for Developability Machine Learning

Input sequences for training machine learning models for biophysical and biochemical property prediction can come from the following illustrative examples:
1) Data on individual sequences where sequences are from:
   a) Discovery efforts using internal libraries; and
   b) Data on sequences made from the literature, such as clinical antibodies, sequences from patents, and the like;
2) Data on pools or collections of sequences, such as:
   a) NGS sequencing on library populations sorted for favorable biophysical properties such as low poly-specificity, low hydrophobicity, high expression, and the like; and
   b) Polyclonal assessment of libraries with known input sequence or composition differences.

Biophysical and biochemical data on the above sequences may include, for example:
1) Poly-specificity measurements using PSR (poly-specificity reagent) and AC-SINS (affinity-captured self-interaction nanoparticle spectroscopy);
2) Hydrophobicity measured using HIC (hydrophobic interaction chromatography) retention time;
3) Melting temperature;
4) SEC (size-exclusion chromatography) monomer percentage and retention time;
5) Chemical stability data under different stress conditions to identify deamidation, isomerization, oxidation, and fragmentation using tryptic peptide mapping; and
6) Sequence enrichment or depletion in positively or negatively selected populations compared mutually, or to input frequencies

$${E}_{property}^{seg} = \frac{{p}_{output population}^{seg}}{{p}_{input}^{seg}}$$

where property can be expression, poly-specificity, or the like, and p is the frequency of the sequence or sequence motif in the population.

### Section IV. Machine Learning and Statistical Models for Sequence Developability

In certain embodiments, the following machine learning models are developed based on input sequences and characterization data as detailed above:
a. Logistic regression models to derive amino-acid coefficients to predict poly-specificity and hydrophobicity for individual positions, CDRs and FRs;
b. Tree-based models such as randomforest and XGBoost to predict biophysical properties from sequence;
c. Deep-learning models using neural networks to predict biophysical properties from sequence;
d. Statistical models to evaluate bias to select sequences with low bias; and
e. Hierarchical statistics to predict risk of chemical modifications as a function of sequence motifs (where “motif” is defined as potentially modified amino acid and immediately succeeding N+1 amino acid) at a specific position and region (CDRH1, CDRH2, CDRH3, CDRL1, CDRL2, CDRL3, HFR, LFR), based on the rate(s) of prior experimentally observed modifications of a motif at a position, region, or anywhere in an antibody sequence. Statistics are hierarchical, as the statistic most specific to a prediction of interest with enough prior observations is used.

Each of these machine learning models is described in more detail, as follows.

a. Logistic Regression

Logistic regression may be performed, for example, using the methodology described in Jain T, Bioinformatics, 33, 3758, 2017, PMID: 28961999. The results from these models are region-specific amino-acid coefficients for prediction of poor developability properties such as delayed retention time in HIC, high poly-specificity, expression, and the like. The following formula is used:

$$log ( \frac{{P}_{clean}}{1 - {P}_{clean}} ) = α + {\sum }_{R ∈ ( CDRs , FR )} {\sum }_{i ∈ A A} {A}_{i}^{R} {ϕ}_{i}^{R}$$

where Aᵢ^{R} is the sum of solvent-exposed side-chain area in region R for residue-type i, determined as described in Jain T, Bioinformatics, 33, 3758, 2017, PMID: 28961999, or from 3D structure, and the like. Region-specific amino-acid coefficients of ϕᵢ^{R} are estimated using logistic regression on data where P_{clean} indicates the likelihood of a sequence with desirable developability characteristics. The formula above can be modified to estimate position-specific coefficients by replacing the outer sum over R with an outer sum over individual positions instead.

As an alternative to point estimates of coefficients, ϕᵢ^{R}, generalized additive models (GAM) were also investigated to fit continuous splines or polynomial coefficients.

b. Tree-Based Regression and Classification Models

Given a property or metric of interest, regression and classification methods (for example, tree-based methods such as randomforest or XGBoost) are used as inputs to train neural network or other machine learning models to predict such properties for novel sequences, segments of sequence, and/or individual amino acids within a sequence. In this instance:
Property˜f(sequence descriptors)
   where sequence (or segment) descriptors include one or more of the following:
1) Sequence or segment length;
2) Hydrophobicity score from logistic regression;
3) Poly-specificity score from logistic regression;
4) Solvent accessibility from neural network prediction;
5) Local structural properties, such as distance between c-alpha atoms of neighboring amino acids or protein backbone phi or psi torsion, as determined by experimental structure and/or structural prediction e.g., prediction via tools such as AlphaFold, IgFold, or direct prediction of atom positions via trained neural network positions;
6) Number of positively (Arg, Lys, His), negatively (Asp, Glu), and total charged residues;
7) Number of aromatic (Phe, Tyr, Trp), aliphatic (Ala, Leu, Val, Ile, Met, Cys), and polar (Asn, Gln, Thr, Ser, His, Gly) residues. Certain amino-acids, such as Gly, His, are considered individually or as part of a class of amino-acids;
8) Identify of “motif” amino acid (immediately succeeding N+1 amino acid after an amino acid of interest), or the class of the motif amino acid, including class it correlates to, protein conformational flexibility, size, chemical, or biophysical property of the motif amino acid;
9) Adjacent primary structure context around an amino acid, for example, the sequence of the X1 amino acids before (e.g., 10) and X2 amino acids after (e.g., 10) a residue of interest;
10) The position of an amino acid within an antibody sequence, for example, as defined by the Chothia or other numbering scheme which enumerates amino acids according to their structural position;
11) The position of an amino acid within a complementary determining region (CDR);
12) Structural conformation associated with framework or CDR sequence, such as those defined by canonical structure clustering (an illustrative example: proline at position L95 in length 9 CDRL3s);
13) Closest wild type germline, and originating species of that wild type antibody sequence;
14) Number of mutations away from nearest wild type, germline antibody sequence;
15) Past statistics based on experimental observations of the output property of interest being predicted, such as, but not limited to, observed modification rates at specific positions or regions.

An illustrative calculation of hydrophobicity and poly-specificity scores is performed as follows:
1) Calculate solvent-accessibility for each residue in the sequence from graphical convolution models described earlier, or perform a lookup on pre-calculated values from a database generated by calculations on a set of known structures, Aᵢ^{R};
2) Count the number of amino-acids of a particular type in the sequence Nᵢ^{R}; and
3) Multiply Aᵢ^{R} or Nᵢ^{R} by the coefficients ϕᵢ^{R}, and sum the values to obtain a final score for the sequence of interest.
   c. Deep-Learning Models for Predicting Developability from Sequence

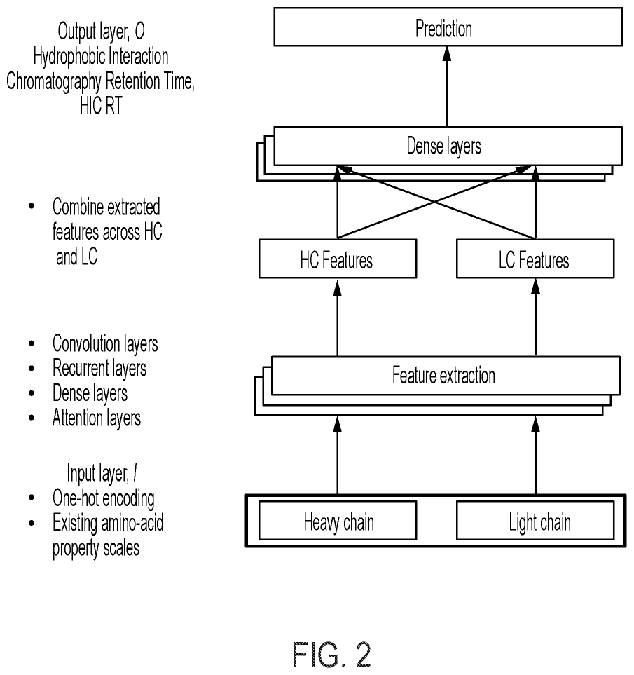

Given a property or metric of interest, deep-learning neural network methods are trained to predict such properties for novel sequences or segments. These models may include an input layer, multiple intermediate feature-extraction layers, and a final output layer, as shown in the schematic diagram of FIG. 2.

Input sequences of different length are processed to the same length for input to the neural network. This can be accomplished by aligning the sequences using a consistent numbering scheme, or by right padding the sequences with the appropriate number of insertions. Subsequently, the sequences are converted into numbers using a one-hot encoding scheme and addition of biophysical and biochemical features using amino-acid property scales, position-specific scoring matrices, and pre-trained sequence embeddings. Descriptors calculated as output from upstream machine learning models/modules, such as the graphical convolution models described herein, may also be calculated from sequence and added to the model inputs.

The model inputs can be adapted to different modalities by adding or subtracting chain information in the input layer. The feature-extraction layers may include one or more of convolution, recurrent (using Long Short-Term Memory (LSTM) units, Gated Recurrent Units (GRU)), self-attention, and/or dense layers.

In one illustrative example, ten-fold cross-validation was used to train the models. In this example, model training was carried out for a maximum of 300 epochs with early termination if improvement was not seen on the test set for more than 10 epochs.

Illustrative pseudo-code for deep-learning models to predict developability is as follows:

| | |
|---|---|
| 7. | xᵢ = Concatenate[onehot encoding, amino - acid property scales, pre - |
| | trained embeddings, position - |
| | specific scoring matrix, multiple sequence alignment, structural |
| | predictions from GCN] |
| 8. | Eᵢ = EmbeddingLayer(xᵢ) |
| 9. | For k in 1: NumFeatureIterations: |
| | Rᵢ = Eᵢ |
| | Cᵢ = Eᵢ |
| | For c in 1: NumConvolutionLayers: |
| | Cᵢ = ConvolutionLayer_{c}(Cᵢ) |
| | For r in 1: NumRecurrentLayers: |
| | Rᵢ = RecurrentLayerᵣ(Rᵢ) |
| | Eᵢ = Concatenate[Rᵢ, Cᵢ] |
| 10. | Dᵢ = Flatten(Eᵢ) |
| | Dᵢ = Concatenate[Dᵢ, Germline, CDR lengths, Species, Mutations |
| | from germline] |
| | For d in 1: NumDenseLayers: |
| | Dᵢ = Dense_{d} [symbol] (Dᵢ) |
| 11. | Oᵢⁿ = Denseⁿ(Dᵢ) |

i ∈ [1 ... L], where L is the sequence length
n ∈ [1 ... N], where n indexes the N output properties to be predicted [symbol]
indicates data missing or illegible when filed

d. Statistical Models for Identifying Sequences with Low Bias

Statistical and datamining approaches are presented herein to identify sequences or sequence motifs that pair equitably across multiple sources of diversities in a library. Given an ideal or target distribution of distinct diversities, proposed motifs are evaluated for their ability to match this distribution with low bias using the Kullback-Leibler divergence metric, for example. Motifs may be a single amino-acid at a position, combinations of amino-acids at different positions, or entire sequences. The Kullback-Leibler divergence metric for a given motif may be computed as follows:

$$KL ( Motif ) = {\sum }_{i ∈ [ D i v 1 , D i v 2 , … ]} P ( i ) log ( \frac{P ( i )}{P ( i \vert Motif )} )$$

where i denotes a type of diversity, P(i) is the ideal or target probability distribution of diversity i, and P(i|Motif) is the conditional probability of the diversity given a sequence. A higher value of KL(Motif) indicates greater departure of P(i|Motif) from P(i). A KL(Motif) value of zero indicates a perfect match between the target and conditional distributions, indicating no bias introduced on account of the sequence or motif.

### Section V. Machine Learning Models for Sequence Patterns and Composition

Auto-regressive deep-learning neural network models may be implemented to learn sequence patterns in a set of curated input training sequences. A set can be comprised of sequences grouped according to desired criteria or characteristics, such as species and germline, preferred developability profile, and the like. The objective is to learn the joint sequence probability distribution over sequences of interest, as follows:

$$P ( Sequence \vert germline ) = {\prod }_{x = 1}^{L = Len of sequence} P ( a {a}_{x} \vert {aa}_{1} , a {a}_{2} , … , {aa}_{x - 1} , germline )$$

In one example, input sequences are padded with one insertion on both sides. These insertions serve as tokens to indicate to the model the beginning and end of the input sequence, and can be subsequently used in the generative step to start novel sequence generation, and detect the end of sequence generation. The padded sequences are converted into numbers using a one-hot encoding scheme, with optional addition of biophysical and biochemical features using amino-acid property scales. The multiple architectures may be investigated by employing intermediate layers containing Long Short-Term Memory (LSTM) units, Gated Recurrent Units (GRU), Dense neurons, Convolution units, and/or Self-attention modules.

In one illustrative example, the input sequence data was split into training: test sets in a 3:1 proportion. In this example, model training was carried out for a maximum of 300 epochs with early termination if improvement was not seen on the test set for more than 10 epochs.

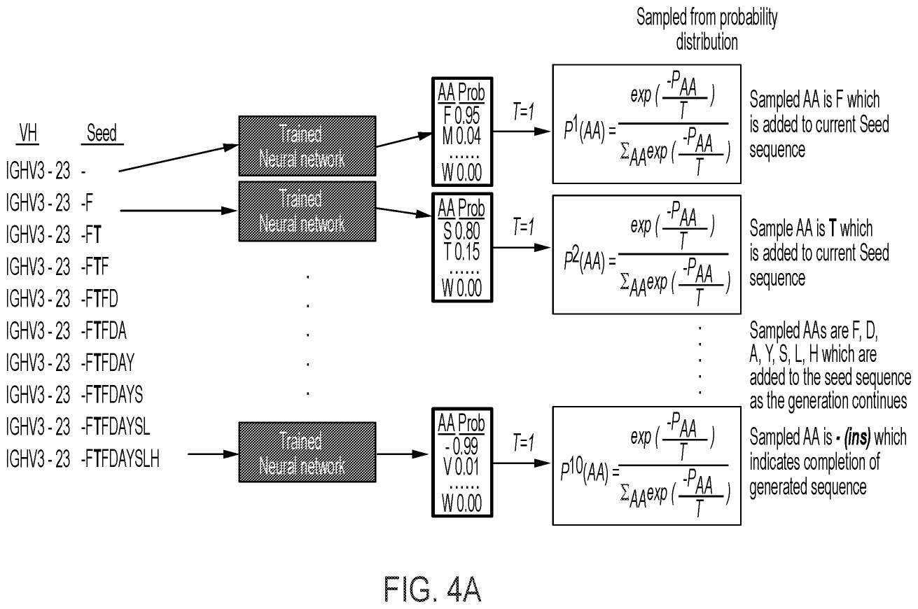

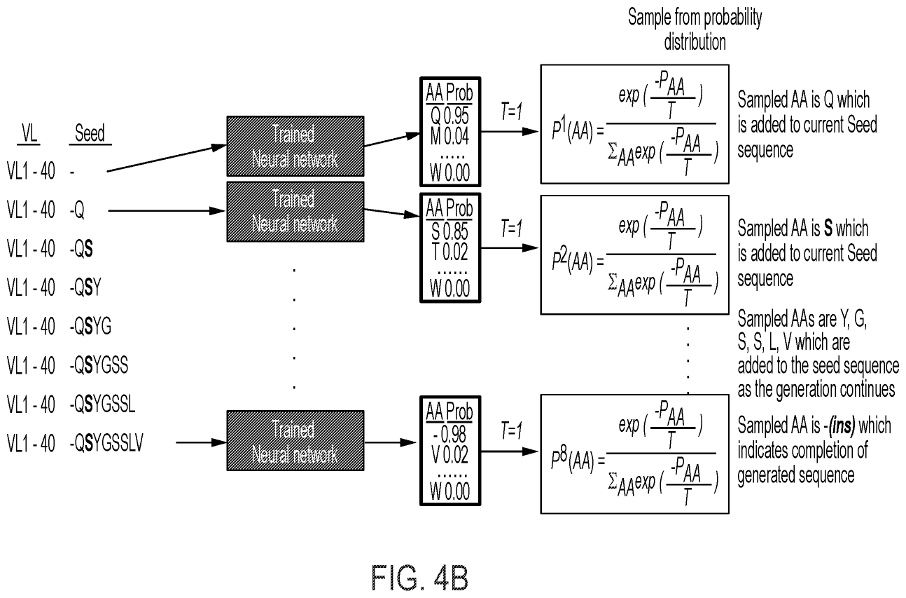

The trained models can be subsequently run in a generative mode by initiating them with an input seed sequence, for example, an alphanumeric (other than the amino acid symbols), hyphen, or other symbol of length one. For example, in the illustrative schematic of FIG. 4, this is a hyphen, “-”, which acts as an artificial construct to teach the model when the H1 starts and when it ends. The current seed sequence is updated by appending amino acids sampled from the probabilities predicted by the model. The generative process is terminated when an insertion is predicted which indicates an end to the sampled sequence. Additionally, the probability of the generated sequence can also be stored for use in prioritizing sequences for the synthetic library. This results in a set of sequences specific to a target germline.

### Section VI. Obtaining Segments, and Estimating their Usage in Repertoires

Using the collection of V-, D- and J-genes detailed above, or a subset, candidate segments can be generated using methods such as nucleotide deletion, nucleotide addition, nibbling, and the like. For segments to be estimated de novo from the data, wildcard sequences matching any sequence up to lengths 0 to L can be added as placeholders.

A tree-based pruning algorithm (e.g., a match to design algorithm) can be used to match the pools of segments to natural repertoire sequences. Examples are presented in international patent application publications WO 2009/036379 and WO 2012/009568, the texts of which are incorporated by reference herein in their entireties. The usage of each segment is updated based on its use in matching a target pool of sequences.

For multiple segment combination maximally matching a target sequence in the repertoire, each segment's usage can be incremented by the inverse of the number of matching combinations, for example.

In the case where some segment types are wildcards, the portion of the target sequence matching the wildcard may be extracted as a de novo segment and its usage updated as described above.

### Section VII Use of Both (i) an Identified Sequence and (ii) One or More Structurally-Relevant Properties of the Sequence Predicted by a First Model, as Input to a Second Model for Predicting Chemical Stability, Polyspecificity, and Hydrophobicity of a Composition, e.g., in Lieu of Structural Information (e.g., without a Software-Generated Structure)

It is found herein that it is possible to use (i) an identified sequence and (ii) one or more structurally-relevant properties of the sequence predicted by a first model, to predict developability properties such as chemical stability, polyspecificity, and hydrophobicity of sequence compositions, thereby producing novel sequences or segments for inclusion consideration in a synthetic library. In certain embodiments, the predicted structurally-relevant properties can be used in place of the structure itself (e.g., in place of software-determined structure as predicted, for example, by AlphaFold or similar software). The following is an illustrative example showing how this “models feeding into models” concept is used to improve the ability to predict chemical stability, polyspecificity, and hydrophobicity of sequence compositions.

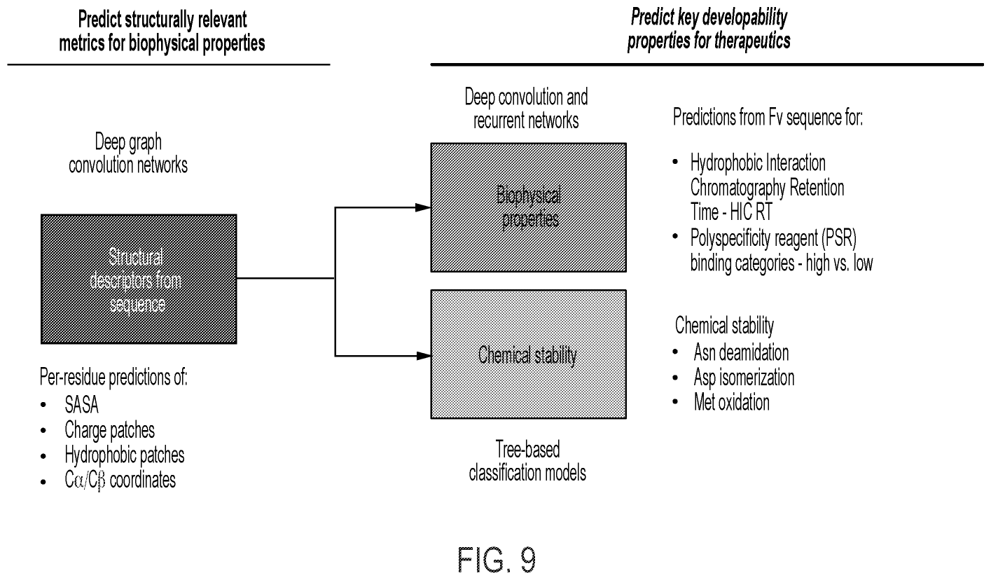

FIG. 9 is a block diagram of a method of using predictions of structurally relevant metrics for biophysical properties in a model to predict key developability properties for therapeutics, according to an illustrative embodiment. At left is represented deep graph convolutional networks which provide structural descriptors from the sequence, for example, per-residue predictions of SASA, charge patches, hydrophobic patches, and Cα/Cβ coordinates. At top right is represented deep convolution and recurrent networks for prediction of biophysical properties, such as predictions from Fv sequence for: hydrophobic interaction chromatography retention time (HIC RT) and polyspecificity reagent (PSR) binding categories (e.g., high vs. low). The structural descriptors from sequence are shown as input in the deep convolution and recurrent networks for prediction of biophysical properties. At bottom right is represented tree-based classification models for prediction of chemical stability properties, such as Asn deamidation, Asp isomerization, and Met oxidation. Again, the structural descriptors from sequence are shown as input in the tree-based classification models for prediction of chemical stability properties.

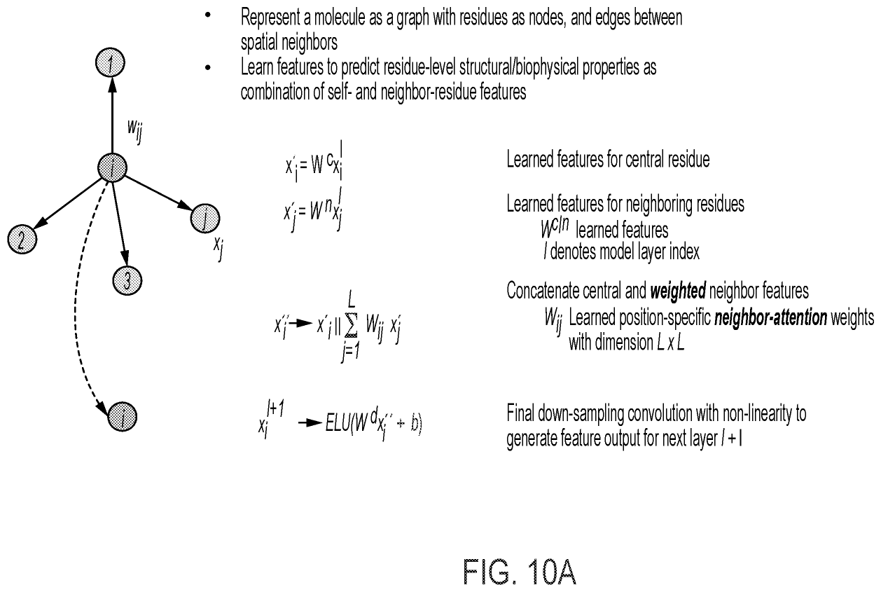

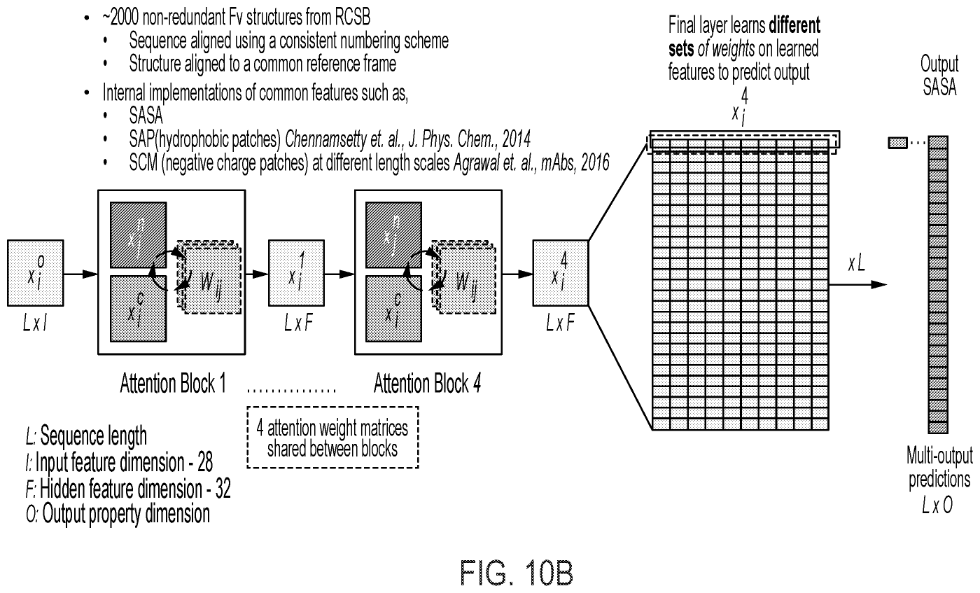

FIGS. 10A and 10B are schematic diagrams illustrating the use of a graphical convolution network (GCN) for residue level prediction of structural descriptors, according to an illustrative embodiment. Sequence is used as input for prediction of structural descriptors such as per-residue predictions of SASA, charge patches, hydrophobic patches, and Cα/Cβ coordinates.

FIG. 10A shows representation of a molecule as a graph with residues as nodes, and edges between spatial neighbors. The graphical convolution network (GCN) can be trained to learn features to predict residue-level structural/biophysical properties as a combination of self- and neighbor-residue features. Learned features for central residue and weighted learned neighbor features are concatenated, then a down-sampling convolution is performed with non-linearity to generate feature output for the next layer.

FIG. 10B is an outline of an illustrative graphic convolution architecture and training data. In this example, there are four attention weight matrices shared between blocks. The final layer learns different sets of weights on learned features to predict output (e.g., solvent accessibility (SASA), which is a key feature of proteins for determining their folding and stability).

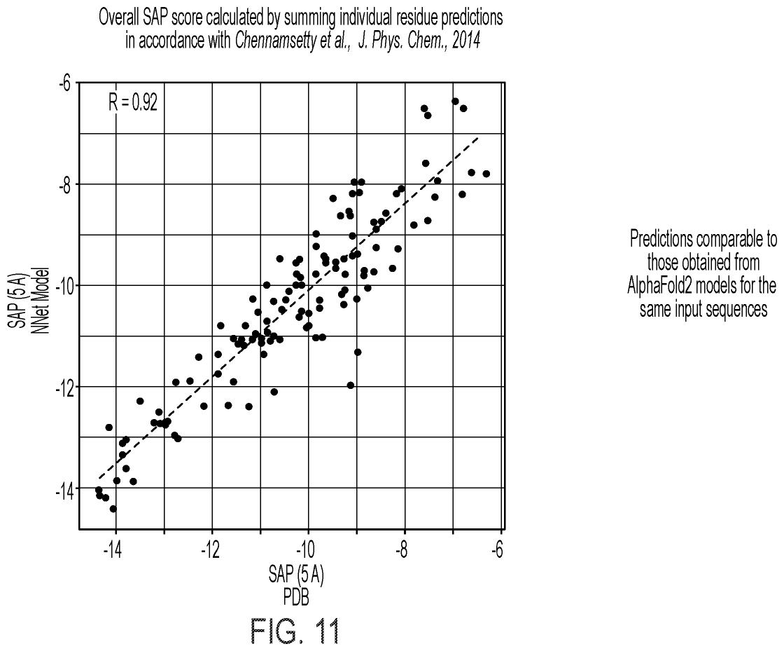

FIG. 11 is a graph showing overall spatial-aggregation-propensity (SAP) score calculated by summing individual residue predictions according to the method above, where the predictions are comparable to those obtained from AlphaFold2 models for the same input sequences.

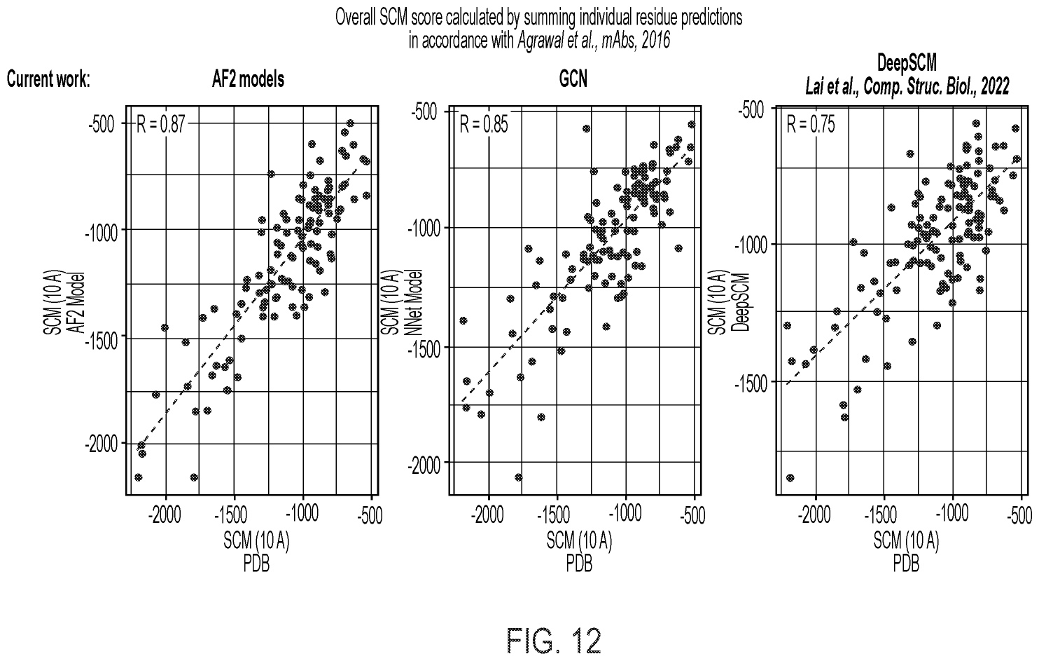

FIG. 12 are graphs showing overall scoring card method (SCM) score calculated by summing individual residue predictions, according to the method above.

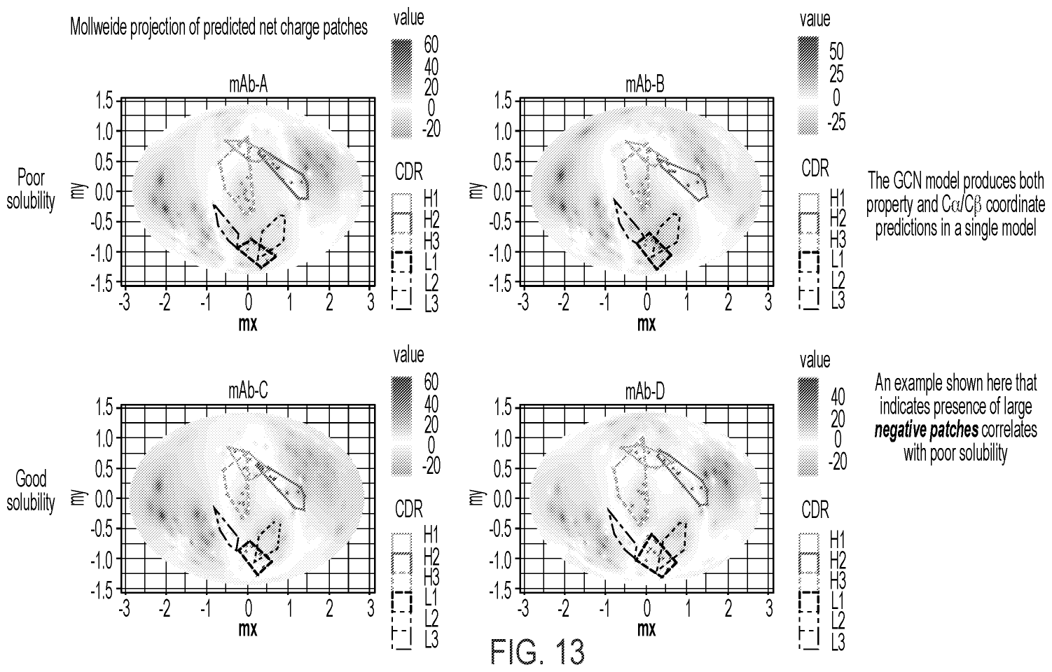

FIG. 13 is a schematic of Mollweide projections of predicted net charge patches, using the method above. The GCN model produces both property and Ca/CB coordinate predictions in a single model. The example shown in FIG. 13 indicates the presence of large negative patches correlates with poor solubility.

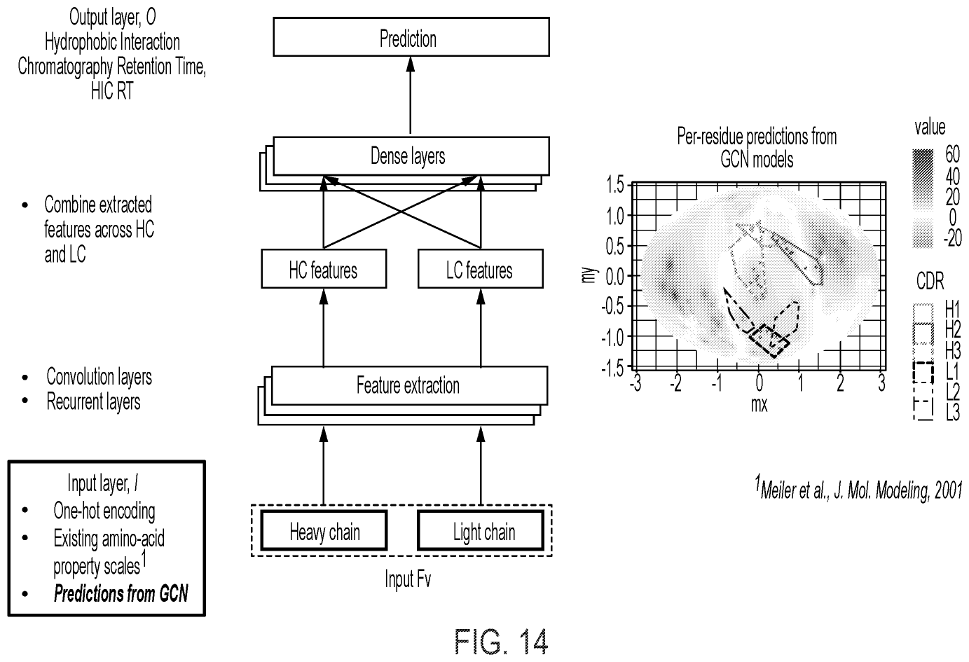

FIG. 14 is a schematic illustrating the use of convolution models and recurrent models for prediction of hydrophobicity and polyspecificity, according to an illustrative embodiment. Patterns in N-mer peptide sequences represent local information or “features”. Interactions between N-mer peptides capture information over longer length scales, as well as between peptides separated along the sequence. In the schematic of FIG. 14, the Input layer, I, uses “input Fv” (antibody fragment sequence) implementing one-hot encoding, for example, existing amino-acid property scales, and predictions of residue-level structural/biophysical properties from the GCN as described above. The next step is feature extraction, where convolution layers of the network architecture learn distinct features over penta-peptides, for example, and recurrent layers learn features over the entire linear sequence. The next step is to combine extracted features across HC and LC. Dense layers learn to globally combine patterns from prior layers. The output layer produces the prediction, e.g., HIC RT or PSR score/category, as described in further detail herein.

### Section VIII. Examples

a. VHH H3 Library Design

### 1. Developability Models for Segment Selection

Fc-linker-VH libraries synthesized with H3 diversity reflecting the human pre-immune repertoire were pressured for expression and poly-specificity using FACS (fluorescence-activated cell sorting). Input libraries, high and low expressing populations, and high and low poly-specificity populations were sequenced using NGS. As outlined in Section II above, the frequency of segment observations in the NGS sequences were used to calculate enrichment scores, E_{high expression}^{seg} and E_{low poly-specificity}^{seg} for the segments. Tree-based machine learning models as described in Section IV were developed (e.g., trained) to enable prediction of desirable segments to enable selection from a pool of de novo and pre-generated segments.

### 2. Estimating Segment Usage, and Inferring De Novo Segments

Pre-generated segments were derived based on the V, D, and J-gene data as described in Section I. For de-novo segment inference, a collection of sequences as detailed in Section I above was used along with the matching algorithm of Section VI above.

The procedure for matching a camelid H3 sequence is outlined as follows:
1) Using the collection of D and J-genes, candidate D- and J-segments were generated;
2) Wildcard N1 segments matching any sequence up to lengths 1 to 9; and
3) Wildcard N2 segments matching any sequence up to lengths 0 to 7.

The CDR H3 sequences from McCoy L E, PLOS Pathogens, 10, e1004552, 2014, PMID: 25522326 and Li X, PLOS ONE, 11, e0161801, 2016, PMID: 27588755 were matched to above pools of segments using methods described in Section VI above to maximize the following metric for match to D- and J-segments:

$$S = Len ( D + J ) {( \frac{Match ( D ) + Match ( J )}{Len ( D ) + Len ( J )} )}^{x} , x \geq 1$$

where Match is the total length of exact matches to the respective segments, and Len is the total length of the matched segments. The portion of the CDR H3 unmatched by D- and J-segments was then used to identify candidate N1 and N2 segments.

The following criteria were used to eliminate matches arising from the above process:
1) The number of mismatches to D- and J-segments is greater than 25% of the length of the D- or J-segments:
2) The total number of mismatches is greater than 5; and
3) Maximal match resulting in Asp or Asn in the last position, or Asn in the penultimate position are removed until a suitable match is found subject to other constraints listed above.

In the case of multiple viable D- and J-segments that maximize S, each segment identified in the match was weighed inversely to number of matches, N.

The outcome from this procedure is a list of usage weights, P, for candidate D- and J-segments generated from a collection of D- and J-genes. Additionally, this procedure produces a list of novel candidates for N1 and N2 segments along with their usage weights, P
p_{usage}^{seg}

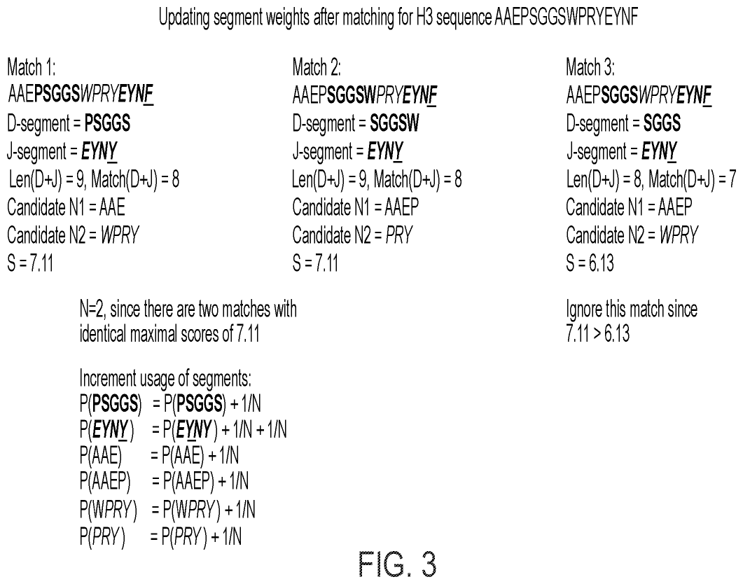

An example for matching CDR H3 sequence AAEPSGGSWPRYEYNF is shown in FIG. 3 using a value of x=2 in the formula for score S.

### 3. Segment Selection for Final Library

The following steps were performed to complete segment selection for the final library:
1) Candidate segments from previous step were input into machine learning models for segment developability to obtain their predicted enrichment scores E_{high expression}^{seg}.
2) All segments with predicted depletion of 40% or more compared to input were filtered, i.e., only retain E_{high expression}^{seg}>0.6.
3) Assign overall importance as the product of predicted expression enrichment and usage weights from match to design:
   I^{seg}=E_{high expression}^{seg}P_{usage}^{seg}
4) A stratified selection of segments was performed by,
   a) For each of the four segment types, select the number of segments for each length based on importance of length

$${I}^{L} = {\sum }_{seg ∈ L} {I}^{s e g} \\ {N}_{t}^{seg} ( L ) = round ( \frac{{Total}_{t} \times {I}^{L}}{{\sum }_{seg ∈ L} {I}^{L}} )$$

where, t denotes the type of segment (N1, D, N2, or J) and Total_t denotes the total number of segments of type t to be selected in the final design; and
b) Next, within each length, select the top Nₜ^{seg}(L) segments by importance score, I^{seg}.

Once segments were selected in this manner, a representative combinatorial library was sampled in silico. This library was evaluated for predicted biophysical characteristics such as poly-specificity and hydrophobicity using models described above. Additionally, sequences from the natural repertoire were also assessed for these properties, and a comparison was made with the novel synthetic design.

Note, the principles and examples disclosed herein for library design for VHH antibodies (or nanobodies) can apply to library design involving other antibody parts, such as light chain framework regions (LC FRs), light chain complementarity-determining regions (LC CDRs), heavy chain framework regions (HC FRs), and others. As used herein, VHH refers to the antigen binding fragment (i.e., variable domain) of a heavy chain-only antibody, e.g. a camelid heavy chain-only antibody.

b. Example for Vλ L3 Library Design

As detailed in Section I, sequence for human Vλ germlines were collected from internal databases, and externa sources such as OAS sequence database, literature, and patent filings.

The observed sequences were split into left- and right-pieces to mimic V-J recombination for CDR L3. Models were built for the collection of CDR L3s, for individual left-sequences, and right-sequences using the methods outlined above.

Once the models were generated to capture sequence compositions and correlations in the input set of sequences, they were run in a generative mode to produce novel sequences or segments for consideration in a synthetic library. An example sequence generation is shown in FIG. 4B. The value of T in the sampling process in FIG. 4B can be increased (decreased) to generate sequences closer (further) from the sequence sets used to train the models.

The final selection of germline-specific sequences was performed in the following manner:
1) Obtain probability of a generated sequence from the generative model;
2) Evaluate poly-specificity and hydrophobicity scores based on CDR-specific amino-acid coefficients derived using logistic regression models or neural network models as detailed in Section IV above. The poly-specificity and hydrophobicity scores were converted into percentile ranks in increments of 5% with lower numbers indicating favorable properties;
3) Evaluate the probability for chemical modifications in the sequence from the neural network or tree-based regression or classification models as described in Section IV;
4) For the generated sequences, calculate number of mutations from germline over the entire sequence, and over the preferentially antigen-contacting residues;
5) The sequence probability from the generative model, mutation information, and predicted poly-specificity, hydrophobicity, and chemical stability scores (e.g., developability ranks) are converted into a priority score for a sequence; and
6) Select top sequences or draw random samples based on their priority scores.

Based on factors such as germline diversity, length distribution, and the like, required in the final library, different proportions of prioritized sequences from germline-specific libraries can be pooled together for a final synthetic library.

c. Example for CDR H1 H2 Library Design

As detailed in Section I above, sequences from human IGHV3 family germlines were collected from external sources such as OAS sequence database, internal databases, literature, and patent filings. Sequences from NGS datasets for llama and camelid were processed from literature studies.

These sequences were renumbered and CDR H1 and H2 sequences were extracted. The subsequent process for training models to learn patterns and running the models in a generative mode follows the process for VA L3 library design.

For example, germline specific models were built for the collection of CDRs H1 and H2, using the methods outlined above.

Once the models were generated to capture sequence compositions and correlations in the input set of sequences, they were run in a generative mode to produce novel sequences for consideration in a synthetic library. An example sequence generation is shown in FIG. 4A. The value of T in the sampling process in FIG. 4A can be increased (decreased) to generate sequences closer (further) from the sequence sets used to train the models.

The final selection of germline-specific sequences was performed in the following manner:
7) Obtain probability of a generated sequence from the generative model;
8) Evaluate poly-specificity and hydrophobicity scores based on CDR-specific amino-acid coefficients derived using logistic regression models or neural network models as detailed in Section III above. The poly-specificity and hydrophobicity scores were converted into percentile ranks in increments of 5% with lower numbers indicating favorable properties;
9) For the generated sequences, calculate number of mutations from germline over the entire sequence, and over the preferentially antigen-contacting residues;
10) The sequence probability from the generative model, mutation information, and developability ranks are converted into a priority score for a sequence; and
11) Select top sequences or draw random samples based on their priority scores.

Based on factors such as germline diversity, length distribution, and the like, required in the final library, different proportions of prioritized sequences from germline-specific libraries can be pooled together for a final synthetic library.

The subsequent process for training models to learn patterns and running the models in a generative mode follows the process for Vλ L3 library design detailed above.

d. Example for Vκ L3 Sequence Design

Data regarding antibodies with suitable developability properties and known heavy and light chain sequences were collected, aligned, renumbered, annotated with germline information. Subsequently, CDR L3s were extracted and amino acids at position L89 to L97 were tabulated.

Referring to notation in Section IV d above, each diversity set i corresponded to sequences belonging to human heavy chain germline VHᵢ. The target distribution P(i) was set as the frequency of sequences belonging to germline VHᵢ. For CDR L3s belonging to a Vκ germline family, the Kullback-Leibler divergence was calculated as a function of L3 position and amino-acid. For example, the calculation of the KL divergence for Alanine at position L91 for Vk1-39 starts by defining the motif as (L91A, Vκ1-39), as follows:

$$KL ( L 91 A , V κ 1 - 39 ) = {\sum }_{i ∈ [ VH germlines ]} P ( i ) log ( \frac{P ( i )}{P ( i \vert L 91 A , V κ 1 - 39 )} )$$

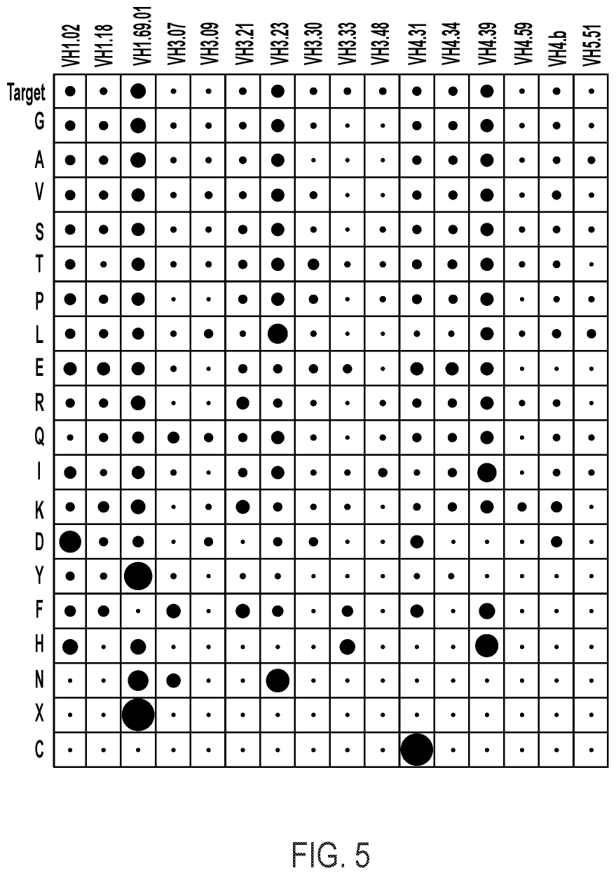

An example distribution used for calculating KL(L91A, Vκ1-39) is shown in FIG. 5 with size of the dots proportional to the probability of the motif seen with VHᵢ. The KL divergence can be similarly defined for higher order motifs covering multiple positions and amino acids at those positions.

The KL calculation for single amino-acid choices at all positions results in a 2-dimensional table with rows indicating position, columns indicating amino acids, and the KL metric as the numeric value in the table. An additional 2-dimensional table was also constructed with the same rows and columns but containing the counts of the amino acids seen at each position.

These tables were used to select sequences from a larger set of CDR L3 sequences using the following procedure:
1. From the tabulated count and calculated KL scores, filter sequences with individual amino-acid choices at a position for low occurrence or high KL score e.g., filter rare or highly biased choices; and
2. Score the remaining sequences by summing the KL score and the tabulated position-specific amino-acid counts in the sequence. Sequences are prioritized by the following two calculated metrics:
   a. The top sequences by descending order of counts; and
   b. The top sequences with the ascending order of summed KL scores.

Different proportions of sequences arising from criteria 2a. and 2b can be used to select the desired number of sequences in the library.

### Software, Computer System, and Network Environment

Certain embodiments described herein make use of computer algorithms in the form of software instructions executed by a computer processor. In certain embodiments, the software instructions include a machine learning module, also referred to herein as artificial intelligence software. As used herein, a machine learning module refers to a computer implemented process (e.g., a software function) that implements one or more specific machine learning algorithms, such as an artificial neural network (ANN), a convolutional neural network (CNN), random forest, decision trees, support vector machines, and the like, in order to determine, for a given input, one or more output values. In certain embodiments, the input comprises alphanumeric data which can include numbers, words, phrases, or lengthier strings, for example. In certain embodiments, the one or more output values comprise values representing numeric values, words, phrases, or other alphanumeric strings. In certain embodiments, the one or more output values comprise an identification of one or more response strings (e.g., selected from a database).

For example, a machine learning module may receive as input a textual string (e.g., entered by a human user, for example) and generate various outputs. For example, the machine learning module may automatically analyze the input alphanumeric string(s) to determine output values classifying a content of the text (e.g., an intent), e.g., as in natural language understanding (NLU). In certain embodiments, a textual string is analyzed to generate and/or retrieve an output alphanumeric string. For example, a machine learning module may be (or include) natural language processing (NLP) software.

In certain embodiments, machine learning modules implementing machine learning techniques are trained, for example using datasets that include categories of data described herein. Such training may be used to determine various parameters of machine learning algorithms implemented by a machine learning module, such as weights associated with layers in neural networks. In certain embodiments, once a machine learning module is trained, e.g., to accomplish a specific task such as identifying certain response strings, values of determined parameters are fixed and the (e.g., unchanging, static) machine learning module is used to process new data (e.g., different from the training data) and accomplish its trained task without further updates to its parameters (e.g., the machine learning module does not receive feedback and/or updates). In certain embodiments, machine learning modules may receive feedback, e.g., based on user review of accuracy, and such feedback may be used as additional training data, to dynamically update the machine learning module. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of a ANN module (e.g., CNN) may be carried out via specialized hardware (e.g., via an application specific integrated circuit (ASIC)).

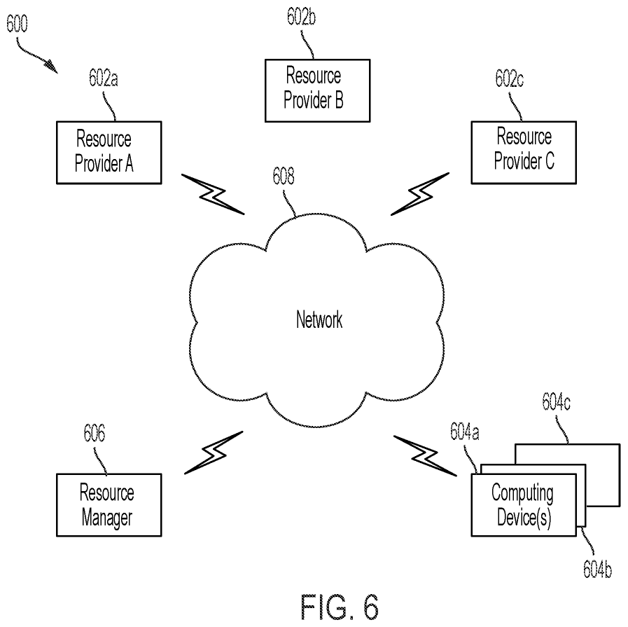

As shown in FIG. 6, an implementation of a network environment 600 for use in providing systems, methods, and architectures as described herein is shown and described. In brief overview, referring now to FIG. 6, a block diagram of an exemplary cloud computing environment 600 is shown and described. The cloud computing environment 600 may include one or more resource providers 602a, 602b, 602c (collectively, 602). Each resource provider 602 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 602 may be connected to any other resource provider 602 in the cloud computing environment 600. In some implementations, the resource providers 602 may be connected over a computer network 608. Each resource provider 602 may be connected to one or more computing device 604a, 604b, 604c (collectively, 604), over the computer network 608.

The cloud computing environment 600 may include a resource manager 606. The resource manager 606 may be connected to the resource providers 602 and the computing devices 604 over the computer network 608. In some implementations, the resource manager 606 may facilitate the provision of computing resources by one or more resource providers 602 to one or more computing devices 604. The resource manager 606 may receive a request for a computing resource from a particular computing device 604. The resource manager 606 may identify one or more resource providers 602 capable of providing the computing resource requested by the computing device 604. The resource manager 606 may select a resource provider 602 to provide the computing resource. The resource manager 606 may facilitate a connection between the resource provider 602 and a particular computing device 604. In some implementations, the resource manager 606 may establish a connection between a particular resource provider 602 and a particular computing device 604. In some implementations, the resource manager 606 may redirect a particular computing device 604 to a particular resource provider 602 with the requested computing resource.

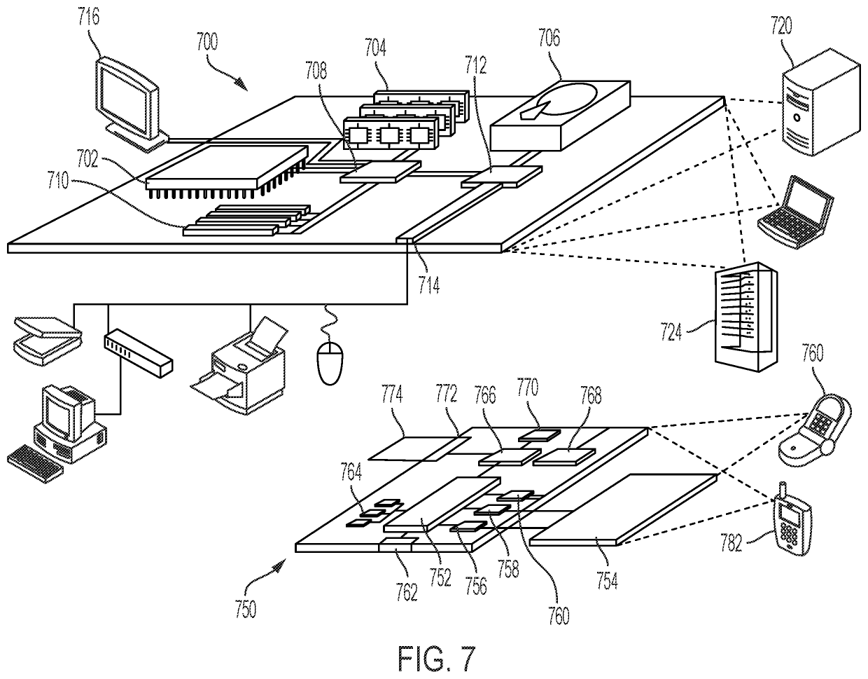

FIG. 7 shows an example of a computing device 700 and a mobile computing device 750 that can be used to implement the techniques described in this disclosure. The computing device 700 is intended to represent various forms of digital computers, such as laptops, desktops, workstations, personal digital assistants, servers, blade servers, mainframes, and other appropriate computers. The mobile computing device 750 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 700 includes a processor 702, a memory 704, a storage device 706, a high-speed interface 708 connecting to the memory 704 and multiple high-speed expansion ports 710, and a low-speed interface 712 connecting to a low-speed expansion port 714 and the storage device 706. Each of the processor 702, the memory 704, the storage device 706, the high-speed interface 708, the high-speed expansion ports 710, and the low-speed interface 712, are interconnected using various busses, and may be mounted on a common motherboard or in other manners as appropriate. The processor 702 can process instructions for execution within the computing device 700, including instructions stored in the memory 704 or on the storage device 706 to display graphical information for a GUI on an external input/output device, such as a display 716 coupled to the high-speed interface 708. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 704 stores information within the computing device 700. In some implementations, the memory 704 is a volatile memory unit or units. In some implementations, the memory 704 is a non-volatile memory unit or units. The memory 704 may also be another form of computer-readable medium, such as a magnetic or optical disk.

The storage device 706 is capable of providing mass storage for the computing device 700. In some implementations, the storage device 706 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 702), perform one or more methods, such as those described above. The instructions can also be stored by one or more storage devices such as computer- or machine-readable mediums (for example, the memory 704, the storage device 706, or memory on the processor 702).

The high-speed interface 708 manages bandwidth-intensive operations for the computing device 700, while the low-speed interface 712 manages lower bandwidth-intensive operations. Such allocation of functions is an example only. In some implementations, the high-speed interface 708 is coupled to the memory 704, the display 716 (e.g., through a graphics processor or accelerator), and to the high-speed expansion ports 710, which may accept various expansion cards (not shown). In the implementation, the low-speed interface 712 is coupled to the storage device 706 and the low-speed expansion port 714. The low-speed expansion port 714, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 700 may be implemented in a number of different forms, as shown in the figure. For example, it may be implemented as a standard server 720, or multiple times in a group of such servers. In addition, it may be implemented in a personal computer such as a laptop computer 722. It may also be implemented as part of a rack server system 724. Alternatively, components from the computing device 700 may be combined with other components in a mobile device (not shown), such as a mobile computing device 750. Each of such devices may contain one or more of the computing device 700 and the mobile computing device 750, and an entire system may be made up of multiple computing devices communicating with each other.

The mobile computing device 750 includes a processor 752, a memory 764, an input/output device such as a display 754, a communication interface 766, and a transceiver 768, among other components. The mobile computing device 750 may also be provided with a storage device, such as a micro-drive or other device, to provide additional storage. Each of the processor 752, the memory 764, the display 754, the communication interface 766, and the transceiver 768, are interconnected using various buses, and several of the components may be mounted on a common motherboard or in other manners as appropriate.

The processor 752 can execute instructions within the mobile computing device 750, including instructions stored in the memory 764. The processor 752 may be implemented as a chipset of chips that include separate and multiple analog and digital processors. The processor 752 may provide, for example, for coordination of the other components of the mobile computing device 750, such as control of user interfaces, applications run by the mobile computing device 750, and wireless communication by the mobile computing device 750.

The processor 752 may communicate with a user through a control interface 758 and a display interface 756 coupled to the display 754. The display 754 may be, for example, a TFT (Thin-Film-Transistor Liquid Crystal Display) display or an OLED (Organic Light Emitting Diode) display, or other appropriate display technology. The display interface 756 may comprise appropriate circuitry for driving the display 754 to present graphical and other information to a user. The control interface 758 may receive commands from a user and convert them for submission to the processor 752. In addition, an external interface 762 may provide communication with the processor 752, so as to enable near area communication of the mobile computing device 750 with other devices. The external interface 762 may provide, for example, for wired communication in some implementations, or for wireless communication in other implementations, and multiple interfaces may also be used.

The memory 764 stores information within the mobile computing device 750. The memory 764 can be implemented as one or more of a computer-readable medium or media, a volatile memory unit or units, or a non-volatile memory unit or units. An expansion memory 774 may also be provided and connected to the mobile computing device 750 through an expansion interface 772, which may include, for example, a SIMM (Single In Line Memory Module) card interface. The expansion memory 774 may provide extra storage space for the mobile computing device 750, or may also store applications or other information for the mobile computing device 750. Specifically, the expansion memory 774 may include instructions to carry out or supplement the processes described above, and may include secure information also. Thus, for example, the expansion memory 774 may be provide as a security module for the mobile computing device 750, and may be programmed with instructions that permit secure use of the mobile computing device 750. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 752), perform one or more methods, such as those described above. The instructions can also be stored by one or more storage devices, such as one or more computer- or machine-readable mediums (for example, the memory 764, the expansion memory 774, or memory on the processor 752). In some implementations, the instructions can be received in a propagated signal, for example, over the transceiver 768 or the external interface 762.

The mobile computing device 750 may communicate wirelessly through the communication interface 766, which may include digital signal processing circuitry where necessary. The communication interface 766 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 768 using a radio-frequency. In addition, short-range communication may occur, such as using a Bluetooth®, Wi-Fi™, or other such transceiver (not shown). In addition, a GPS (Global Positioning System) receiver module 770 may provide additional navigation- and location-related wireless data to the mobile computing device 750, which may be used as appropriate by applications running on the mobile computing device 750.

The mobile computing device 750 may also communicate audibly using an audio codec 760, which may receive spoken information from a user and convert it to usable digital information. The audio codec 760 may likewise generate audible sound for a user, such as through a speaker, e.g., in a handset of the mobile computing device 750. Such sound may include sound from voice telephone calls, may include recorded sound (e.g., voice messages, music files, etc.) and may also include sound generated by applications operating on the mobile computing device 750.

The mobile computing device 750 may be implemented in a number of different forms, as shown in the figure. For example, it may be implemented as a cellular telephone 780. It may also be implemented as part of a smart-phone 782, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, certain modules described herein can be separated, combined or incorporated into single or combined modules. Any modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A system for constructing (e.g., designing) an antibody library, the system comprising:
a processor of a computing device; and
a memory having instructions stored thereon, wherein the instructions, when executed by the processor, cause the processor to perform one or more of (i), (ii), (iii), (iv), (v), (vi), and (vii) as follows:
(i) develop (e.g., train) a first machine learning model using input sequences and characterization data [e.g., (a) train a logistic regression model to derive amino-acid coefficients to predict poly-specificity and hydrophobicity for individual complementarity-determining regions (CDRs) and/or framework regions (FRs); and/or (b) train a tree-based model (e.g., randomforest or XGBoost) to predict one or more biophysical properties and/or one or more chemical stability properties from a sequence; and/or (c) train a deep-learning model comprising neural networks to predict one or more biophysical properties and/or one or more chemical stability properties from a sequence (e.g., wherein the model comprises an input layer, multiple intermediate feature-extraction layers, and a final output layer); and/or (d) create a statistical model to evaluate bias to select sequences with low bias; and/or (e) develop hierarchical statistics to predict risk of chemical modification as a function of sequence motifs at a specific position and region (e.g., H1, H2, H3, L1, L2, L3, HFR, LFR)];
(ii) use the first machine learning model in (i) to predict desirable segments (e.g., segments with favorable predicted expression enrichment) to enable selection of segments from a pool of de novo and/or pre-generated segments;
(iii) process a set of input sequences prior to selection and/or use in training the first machine learning model in (i), wherein processing the set of input sequences comprises one or more of: (a) eliminating chemical liability sites by modifying the sequence, (b) for CDR H3, splitting the sequence into segments to mimic VDJ recombination, (c) for CDR L3, splitting the sequence into segments to mimic VJ recombination, and (d) annotating V-regions and CDRs (H1, H2, L3) with number of mutations from germline;
(iv) train a machine learning model for biophysical and/or biochemical property prediction (e.g., using data on a set of input sequences sorted for favorable biophysical properties, e.g., low poly-specificity, low hydrophobicity, and/or high expression);
(v) use the machine learning model for biophysical and/or biochemical property prediction in (iv) to predict one or more biophysical and/or biochemical properties from a sequence (e.g., poly-specificity, hydrophobicity, melting temperature, SEC monomer percentage, retention time, chemical stability data, and/or a measure of sequence enrichment or depletion;
(vi) develop (e.g., train) an auto-regressive deep-learning neural network model to learn a joint sequence probability distribution over sequences of interest for specific germlines for different species; and
(vii) use the neural network model in (vi) to capture sequence compositions and/or correlations from an input set of sequences and produce novel sequences or segments for consideration in a synthetic library.

2. A system for constructing an antibody library, the system comprising:
a processor of a computing device; and
a memory having instructions stored thereon, wherein the instructions, when executed by the processor, cause the processor to process a set of input sequences to generate a collection of final antibody library sequences using one or more machine learning models.

3. The system of claim 2, wherein the instructions cause the processor to process (i) each input sequence from the set of input sequences as well as, (ii) for each of the input sequences, per-residue predictions of one or more structurally-relevant properties of the sequence as predicted by a first model (e.g., a graph convolutional network (GCN)), said instructions causing the processor to process (i) and (ii) as input in a second model to predict, as output of the second model, (iii) one or more biophysical properties [e.g., hydrophobic interaction chromatography retention time (HIC RT) and/or polyspecificity reagent (PSR) score and/or PSR binding category] and/or (iv) one or more chemical stability properties [e.g., Asn deamidation, Asp isomerization, and/or Met oxidation] of each of the input sequences, wherein inclusion or exclusion of each sequence in the final antibody library is based at least in part on the output of the second model.

4. The system of claim 3, wherein the per-residue predictions predicted by the first model comprise one or more members selected from the group consisting of (i) a measure of solvent accessibility (SASA), (ii) a measure of charge patches, (iii) a measure of hydrophobic patches, and (iv) Cα/Cβ coordinate predictions.

5. The system of claim 3 or 4, wherein the second model comprises a deep convolution and/or recurrent network (e.g., for prediction of biophysical properties).

6. The system of any one of claims 3 to 5, wherein the second model comprises a tree-based classification model (e.g., for prediction of chemical stability).

7. A method for constructing (e.g., designing) an antibody library, the method comprising using a processor of a computing device to perform one or more of (i), (ii), (iii), (iv), (v), (vi), and (vii) as follows:
(i) developing (e.g., training) a first machine learning model using input sequences and characterization data [e.g., (a) training a logistic regression model to derive amino-acid coefficients to predict poly-specificity and hydrophobicity for individual complementarity-determining regions (CDRs) and/or framework regions (FRs); and/or (b) training a tree-based model (e.g., randomforest or XGBoost) to predict one or more biophysical properties and/or one or more chemical stability properties from a sequence; and/or (c) training a deep-learning model comprising neural networks to predict one or more biophysical properties and/or one or more chemical stability properties from a sequence (e.g., wherein the model comprises an input layer, multiple intermediate feature-extraction layers, and a final output layer); and/or (d) creating a statistical model to evaluate bias to select sequences with low bias; and/or (e) developing hierarchical statistics to predict risk of chemical modification as a function of sequence motifs at a specific position and region (e.g., H1, H2, H3, L1, L2, L3, HFR, LFR)];
(ii) using the first machine learning model in (i) to predict desirable segments (e.g., segments with favorable predicted expression enrichment) to enable selection of segments from a pool of de novo and/or pre-generated segments;
(iii) processing a set of input sequences prior to selection and/or use in training the first machine learning model in (i), wherein processing the set of input sequences comprises one or more of: (a) eliminating chemical liability sites by modifying the sequence, (b) for CDR H3, splitting the sequence into segments to mimic VDJ recombination, (c) for CDR L3, splitting the sequence into segments to mimic VJ recombination, and (d) annotating V-regions and CDRs (H1, H2, L3) with number of mutations from germline;
(iv) training a machine learning model for biophysical and/or biochemical property prediction (e.g., using data on a set of input sequences sorted for favorable biophysical properties, e.g., low poly-specificity, low hydrophobicity, and/or high expression);
(v) using the machine learning model for biophysical and/or biochemical property prediction in (iv) to predict one or more biophysical and/or biochemical properties from a sequence (e.g., poly-specificity, hydrophobicity, melting temperature, SEC monomer percentage, retention time, chemical stability data, and/or a measure of sequence enrichment or depletion);
(vi) developing (e.g., training) an auto-regressive deep-learning neural network model to learn a joint sequence probability distribution over sequences of interest for specific germlines for different species; and
(vii) using the neural network model in (vi) to capture sequence compositions and/or correlations from an input set of sequences and produce novel sequences or segments for consideration in a synthetic library.

8. A method for constructing (e.g., designing) an antibody library, the method comprising:
processing, by a processor of a computing device, a set of input sequences to generate a collection of final antibody library sequences using one or more machine learning models.

9. The method of claim 8, comprising processing, as input in a second model, (i) each input sequence from the set of input sequences as well as (ii) for each of the input sequences, per-residue predictions of one or more structurally-relevant properties of the sequence as predicted by a first model (e.g., a graph convolutional network (GCN)), to produce, as output of the second model, (iii) one or more biophysical properties [e.g., hydrophobic interaction chromatography retention time (HIC RT) and/or polyspecificity reagent (PSR) score and/or PSR binding category] and/or (iv) one or more chemical stability properties [e.g., Asn deamidation, Asp isomerization, and/or Met oxidation] of each of the input sequences, wherein inclusion or exclusion of each sequence in the final antibody library is based at least in part on the output of the second model.

10. The method of claim 9, wherein the per-residue predictions predicted by the first model comprise one or more members selected from the group consisting of (i) a measure of solvent accessibility (SASA), (ii) a measure of charge patches, (iii) a measure of hydrophobic patches, and (iv) Cα/Cβ coordinate predictions.

11. The method of claim 9 or 10, wherein the second model comprises a deep convolution and/or recurrent network (e.g., for prediction of biophysical properties).

12. The method of any one of claims 9 to 11, wherein the second model comprises a tree-based classification model (e.g., for prediction of chemical stability).
